(19) United States
(12) Patent Application Publication
(10) Pub. No.: US 2014/0146936 A1
(43) Pub. Date: May 29, 2014

(54) METHOD AND SYSTEM FOR GATED RADIATION THERAPY

(71) Applicant: GE Medical Systems Global Technology Company, LLC, Waukesha, WI (US)

(72) Inventors: Ping LIU, Beijing (CN); Jiaqin DONG, Beijing (CN); Yilun SHI, Beijing (CN)

(73) Assignee: GE Medical Systems Global Technology Company, LLC, Waukesha, WI (US)

(21) Appl. No.: 14/069,542

(22) Filed: Nov. 1, 2013

(30) Foreign Application Priority Data
Nov. 27, 2012 (CN) 201210489932.8

(51) Int. Cl. A61N 5/10; A61B 6/03

(52) U.S. Cl. CPC A61N 5/1067; A61B 6/032
USPC 378/8

(57) ABSTRACT

A method and system for a gated radiation therapeutic system is provided. The method comprises performing a 4D-CT scan to obtain CT images, and processing the CT images to extract a characteristic signal, wherein the characteristic signal is associated with respiratory phases within the patient's breathing cycle and has a correlation relation with the external signal of the gated radiation therapy. The method further comprises controlling a radiation beam for the gated radiation therapy based on the correlation relation between the characteristic signal and the external signal. The system comprises a 4D-CT scanner for performing a 4D-CT scan to obtain CT images and a characteristic signal extracting component for processing the CT images to extract a characteristic signal. The system further comprises a radiation control component for controlling a radiation beam for the gated radiation therapy based on the correlation relation between the characteristic signal and the external signal.

### TECHNICAL FIELD

The present invention relates to radiation therapy and, more particularly, to a method and system for gated radiation therapy.

### BACKGROUND OF THE INVENTION

With the development of the medical technology, radiation therapy is widely used in the treatment of diseases. Usually, the success of a radiation therapy depends on the accuracy in depicting the pathological tissues. A major problem in the therapy is that the target motion caused by, for example, the patient's breathing may cause artifacts in a conventional free-breathing CT scan. To solve this problem, a four-dimensional (4D) CT technique is developed for depicting the moving target and modeling the target motion.

4D CT is accomplished by over-sampling CT data in each couch and sorting the scanned images into multiple CT volumes corresponding to respiratory phases. Currently, two types of 4D CT are researched, one is external device-based 4D CT (A4D-CT), and the other is device-less 4D CT (D4D-CT). A4D-CT uses an external signal recorded by an external instrument outside a CT scanner for sorting, while D4D-CT is based on the patient internal anatomy for sorting, and its respiratory signal is extracted from CT image features.

In the prior art, there are a large number of papers on 4D-CT scanning and sorting, e.g., the article “4D-CT sorting based on patient internal anatomy” (Phys. Med. Biol. 54 (2009) 4821-4833) written by Ruijiang Li presents a D4D-CT sorting method based on four internal anatomical features. Other introductions on 4D-CT can be found in U.S. patent application Ser. Nos. 10/599,084, 12/290,200, 12/754,824. The contents of these prior arts are incorporated into the present application through citation.

Besides, another pending Chinese patent application (Application No. 201110358351.6, the title of the invention “METHOD, APPARATUS AND SYSTEM FOR D4D-CT IMAGING”) with the same inventor as the inventor of the present invention discloses that the lung expansion and contraction motion vectors, the proportion of the pulmonic cavity to the body may be used for generating respiratory curves and sorting the corresponding CT images (i.e., CT slices). All the contents of this Chinese patent application are incorporated into this application through citation.

In radiation therapy, it is required that the radiation of the predetermined dose should be accurately projected onto the lesion site (e.g., cancer) and the radiation dose projected onto the normal tissues around the lesion site is minimized. The present research has met the above requirements to some extent, but because the patient's breathing may cause the motion of the lesion site, errors often occur in aspects of radiation accuracy and radiation dose.

Usually, using gated radiation therapy can avoid the problems of radiation accuracy and radiation dose in radiation therapy. In the gated radiation therapy, when a patient is in a particular phase of the breathing cycle, the lesion site is in the treatment region onto which a radiation beam is projected, and the radiation beam is emitted only in this particular phase. When the patient breathes in other phases, because the lesion site is in motion, the radiation beam cannot be accurately projected onto the lesion site, so there is no need to project the radiation beam.

Respiratory gated radiation therapy usually includes two modes. In the first mode, an internal surrogate organ is used for detecting the motion of the lesion site, and a real-time imaging system, e.g., an X-ray imaging system, is used to provide the positional information of a tag for indicating the motion of the lesion site. In the second mode, external surrogates are used, and various external surrogates, e.g., a strainmeter bound to the patient's body, an air-bag, a real-time position management (RPM) module, can be used.

In the prior art, there are also a large number of papers on the gated radiation therapy, see, for example, the PCT patent application PCT/US2007/017443. The contents of the prior art are incorporated into the present application through citation.

An advantage of using an external gated system is that it is non-intrusive. However, because the external gated system needs to use an external signal provided by the external surrogates, and external signals can be provided only in A4D-CT scan, the prior art merely contains applications where the result of the A4D-CT scan is used for gated radiation therapy. Moreover, due to the necessity of external devices in the A4D-CT scan required by the gated radiation therapy, there are such problems as high cost, inconvenience of use, etc.

In the gated radiation therapy of the prior art, there are also shortcomings in other aspects. Therefore, there is a need for an improved solution that can improve one or more aspects in the prior art, e.g., to enable the gated radiation therapy to use not only the A4D-CT scan result but also the D4D-CT scan result, to reduce the cost of the gated radiation therapy, and/or increase the efficiency of the gated radiation therapy.

### SUMMARY OF THE INVENTION

Embodiments of the present invention is intended to solve one or more problems existing in the prior art, particularly, to make the gated radiation therapy capable of using both the AD4D-CT and D4D-CT scan results so as to reduce the cost of the gated radiation therapy and/or increase the efficiency of the gated radiation therapy.

According to an embodiment of the present invention, a method for a gated radiation therapeutic system is provided, the method comprising: performing a 4D-CT scan to obtain CT images; processing the CT images to extract a characteristic signal, wherein the characteristic signal is associated with respiratory phases within the patient's breathing cycle, and the characteristic signal has a correlation relation with the external signal of the gated radiation therapy; and controlling a radiation beam for the gated radiation therapy on the basis of the correlation relation between the characteristic signal and the external signal.

According to one embodiment, controlling the radiation beam on the basis of the correlation relation between the characteristic signal and the external signal comprises: sorting the CT images to obtain a 4D-CT image; setting a gated radiation therapeutic system on the basis of the 4D-CT image and the characteristic signal; measuring a current external signal of the gated radiation therapy to determine a current respiratory phase; and controlling the radiation beam on the basis of the current respiratory phase and the correlation relation between the characteristic signal and the external signal.

According to one embodiment, setting the gated radiation therapeutic system on the basis of the 4D-CT image and the characteristic signal comprises: projecting radiation onto only a lesion site within the patient's body when a radiation beam is emitted in a particular respiratory phase indicated by the characteristic signal according to the 4D-CT image, because the precise position of the lesion site in the particular phase can be determined through the 4D-CT image.

According to one embodiment, controlling the radiation beam on the basis of the current respiratory phase comprises: emitting the radiation beam in the current respiratory phase when the current respiratory phase is identical with or adjacent to the particular respiratory phase.

According to one embodiment, the external signal of the gated radiation therapy is a real-time position management (RPM) signal provided by a real-time position management (RPM) module.

According to one embodiment, the characteristic signal indicates a body surface motion of the patient.

According to one embodiment, the body surface motion comprises a body surface height in a selected region of a particular couch of the 4D-CT scan.

According to one embodiment, the body surface height comprises a maximum height value, an average height value, etc. of the patient's body surface height in the selected region.

According to one embodiment, the method for a gated radiation therapeutic system comprises: fixing a tag to the patient's body when the 4D-CT scan is performed; according to the obtained 4D-CT image, a relative position of the tag and the lesion site is determined; according to the determined relative position, the lesion tag is fixed to the body surface above the lesion site; and when the radiation therapy is carried out, the PRM module is arranged at the lesion tag.

According to one embodiment, the correlation relation between the characteristic signal and the external signal of the gated radiation therapy comprises the characteristic signal matches the external signal, etc.

According to one embodiment, the characteristic signal is used for sorting the CT images to obtain a D4D-CT image.

According to one embodiment, the characteristic signal is obtained from one of the following parameters or their combinations: air content, lung area, lung density, body area, etc., lung expansion and contraction motion vectors, the proportion of the pulmonic cavity to the body, etc.

According to an embodiment of the present invention, a system for gated radiation therapy is provided, the system comprising: a 4D-CT scanner for performing a 4D-CT scan to obtain CT images; a characteristic signal extracting component for processing the CT images to extract a characteristic signal, wherein the characteristic signal is associated with respiratory phases within the patient's breathing cycle, and the characteristic signal has a correlation relation with the external signal of the gated radiation therapy; and a radiation control component for controlling a radiation beam for the gated radiation therapy on the basis of the correlation relation between the characteristic signal and the external signal.

According to an embodiment of the present invention, a method for a radiation therapeutic system is provided, the method comprising: performing a 4D medical scan to obtain images; processing the images to extract a characteristic signal, the characteristic signal is associated with respiratory phases in a patient's breathing cycle, and the characteristic signal has a correlation relation with the external signal of the radiation therapy; and controlling a radiation beam for the radiation therapy on the basis of the correlation relation between the characteristic signal and the external signal.

The improved solution of embodiments of the present invention can solve one or more problems existing in the prior art. With the present invention, the gated radiation therapy can use not only the AD4D-CT scan result but also the D4D-CT scan result, and thereby the cost of the gated radiation therapy is reduced and/or the efficiency of the gated radiation therapy is increased.

### DESCRIPTION OF THE DRAWINGS

The advantages, characteristics and features of embodiments of the present invention can be further understood through the description on the specific implementation modes of embodiments of the present invention in conjunction with the drawings, wherein:

FIG. 1 shows a 4D-CT sorting and reconstructing process;

FIG. 2 shows a block diagram of a method for a gated radiation therapeutic system according to one embodiment of the present invention;

FIG. 3 shows a comparison between non-gated radiation therapy and the gated radiation therapy according to one embodiment of the present invention;

FIG. 4 shows that a characteristic signal is obtained by using a body surface motion according to one embodiment of the present invention;

FIG. 5 shows that a RPM module is arranged on the patient's body according to one embodiment of the present invention;

FIG. 6 shows the operable region where the RPM module may be arranged according to one embodiment of the present invention;

FIG. 7 shows the matching of the characteristic signal and the RPM signal when the RPM module is positioned above the lesion site according to one embodiment of the present invention;

FIG. 8 shows the matching of the characteristic signal and the RPM signal when the RPM module is positioned in the operation region, but not above the lesion site according to one embodiment of the present invention; and

FIG. 9 shows a system for a gated radiation therapy according to one embodiment of the present invention.

### DETAILED DESCRIPTION OF THE INVENTION

The present invention will be described in more detail in the following with reference to the drawings, in which the embodiments of the present invention are shown. However, it shall be understood that the present invention may be implemented in other different manners, and is not limited to these specific embodiments. Conversely, the provision of these embodiments is intended to make the disclosure of the present invention more thorough and complete so that the concept of the present invention can be completely understood by those skilled in the art. Throughout this application, identical or similar reference signs represent the same means or unit.

In order to eliminate or reduce the influence of respiratory motion artifacts on CT scan of chest and abdomen and to reflect the motion of tumors in the chest and abdomen over time and achieve the object of accurate diagnosis and treatment, the concept of four-dimensional CT is presented, in which the time factor is considered during the three-dimensional reconstruction of CT scan images, so that a dynamic four-dimensional CT image can be formed.

To take A4D-CT based on external devices for example, the general process of implementing A4D-CT of the chest and abdomen on a CT machine is as follows: when images are being collected, a respiratory monitoring system connected with the CT machine is used for detecting the patient's breathing, and the CT images and a breathing signal are collected at the same time. The collected CT images are marked with time information in the breathing cycle (i.e., phase), and then based on the respective phases, all the CT images are respectively divided into groups and three-dimensional CT images are reconstructed, wherein the three-dimensional images of the phases form a three-dimensional image sequence over time, i.e., 4D-CT. A 4D-CT system mainly uses a pneusometer to measure the patient's breathing amount, an infrared photographic equipment to measure the height difference of the patient's body surface as the patient breathes, or a pressure sensor to measure the pressure difference caused by the patient's breathing. These measurement signals can be converted to breathing signals. Usually, the CT image is collected using a CINE mode, in which the CT images are continuously collected in each couch for a certain period. After the CINE is performed for one couch, another CINE scan is performed for the next couch. The CINE scan is repeated until the whole range that needs to be scanned is covered.

The above A4D-CT reconstructing method requires that in the image collecting process, a breathing detector shall communicate with the CT machine, and that the breathing signals are synchronous with the CT image collection. However, because the signals obtained by monitoring the patient's body surface are not synchronous with the motion of the internal organs within the body and the breathing motion is not exactly repeated during the different breathing cycles, usually the CT images reconstructed from the sorting are not accurate, e.g., mismatching in the direction of Z axis frequently occurs.

D4D-CT scan can avoid the disadvantages of A4D-CT scan to some extent.

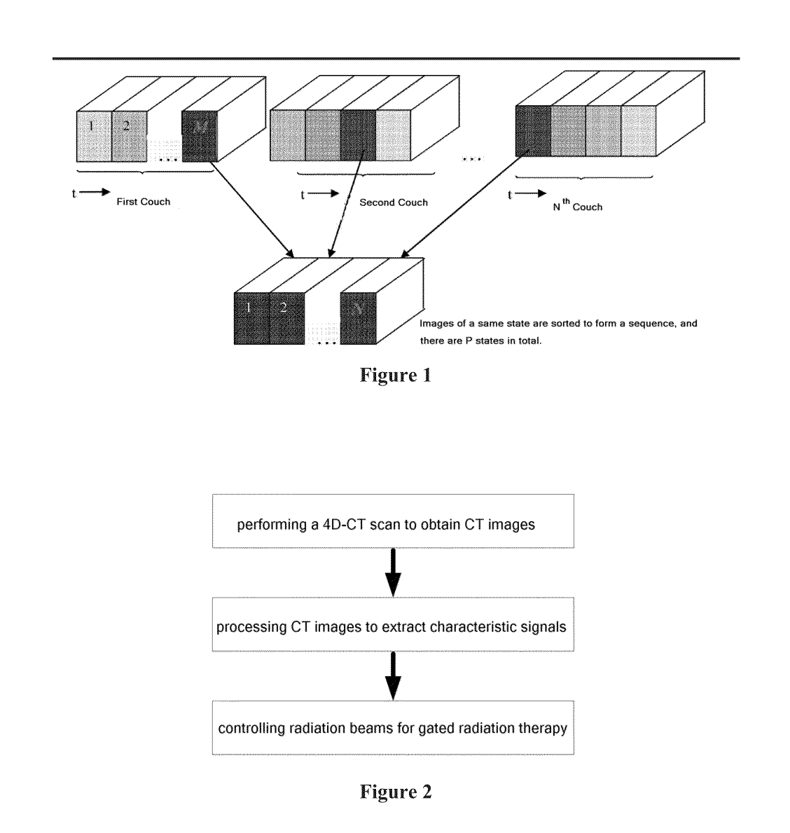

FIG. 1 briefly shows a 4D-CT sorting and reconstructing process. In FIG. 1, there are N couches in total in the CINE scan, and in each couch M times of sampling are performed in respiratory phases of the patient's breathing. New sequences can be formed with the samples in the same phase of N different couches. If P phases are considered in a cycle, then P new sequences can be formed, wherein each new sequence is corresponding to a 3D-CT image of a scan target in a respiratory phase. Usually the whole time for sampling is longer than a breathing cycle of the scan target, so generally the following relationship exists: M>=P. Under a special condition, however, it is possible that M<P.

After the 4D-CT (A4D-CT or D4D-CT) scan, the formed CINE image is selected and divided into a plurality of phase groups. The plurality of phase groups are respectively corresponding to a plurality of respiratory phases of the scan target. Each of the plurality of respiratory phases corresponds respectively to one 3D-CT image of the scan target in one respiratory phase. The process of forming the new sequences or phase groups is called “sorting”.

Breathing signals of the breathing cycle, sorting, etc are well known in D4D-CT imaging, and therefore are not further described.

FIG. 2 shows a method for a gated radiation therapeutic system according to one embodiment of the present invention. The method comprises: performing a 4D-CT scan to obtain CT images (i.e., CT slices); processing the CT images to extract a characteristic signal; and controlling a radiation beam for the gated radiation therapy on the basis of the correlation relation between the characteristic signal and the external signal.

The characteristic signal is associated with respiratory phases within the patient's breathing cycle. Similar to the breathing signals, the characteristic signal indicates characteristic values of a selected feature in respiratory phases of the breathing cycle. Therefore, through measurement of the current characteristic values of the selected feature, it can be determined in which phase of the breathing cycle the patient is according to a curve the characteristic signal. Besides, the characteristic signal has a correlation relation with the external signal of the gated radiation therapy, wherein the external signal may be used in the gated radiation therapy for controlling the gated phase, i.e., to control whether or not to emit the radiation beam according to the phase of the external signal.

The correlation relation includes, but is not limited to the following: the characteristic signal is associated with the external signal used for the gated radiation therapy, e.g., they are completely identical or almost identical, or the characteristic signal and the breathing signals of the external signal are identical or almost identical (e.g., the amplitudes are not identical, but the waveform shapes are identical or almost identical), or the phase of the characteristic signal can be determined based on the phase of the external signal (or vice versa), etc. It shall be understood that the characteristic signal can be extracted before the CT images are sorted or after the CT images are sorted.

Besides, because different external signal types may be used during the gated therapy, a most suitable internal image feature may be selected based on the external signal to generate a characteristic signal. For example, when the external signal is provided by a respiratory binding strip around the body, the characteristic signal obtained by using the body area feature can be well associated with (even identical with) the external signal.

However, it will be understood that even if the characteristic signal is not generated based on the most suitable internal feature, it can be still used for the gated radiation therapy as long as it has a correlation relation with the external signal of the gated radiation therapy.

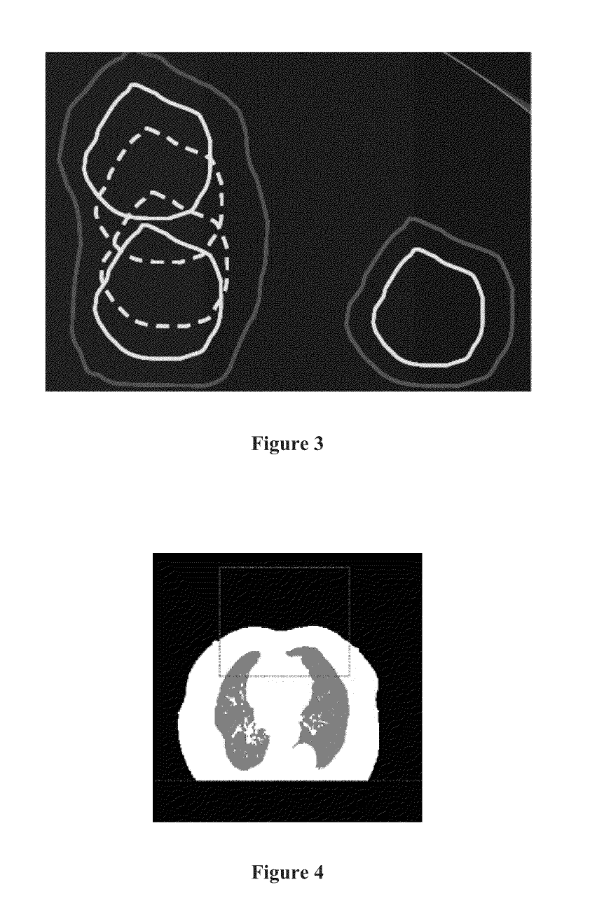

FIG. 3 shows a comparison between non-gated radiation therapy and the gated radiation therapy according to one embodiment of the present invention. The left side of FIG. 3 shows the non-gated radiation therapy, wherein the radiation therapy is directed at the whole motion region of the lesion site. As shown in the figure, in the non-gated radiation therapy, the normal tissues in a larger area around the lesion site are exposed to radiation. Conversely, as shown in the right side of FIG. 3, in the gated radiation therapy, radiation can be accurately projected onto the lesion site and the area of the normal tissues subjected to the radiation around the lesion site is minimized.

According to one embodiment of the present invention, controlling the radiation beam on the basis of the correlation relation between the characteristic signal and the external signal comprises: sorting the CT images to obtain a 4D-CT image; setting the gated radiation therapeutic system on the basis of the obtained 4D-CT image; measuring a current external signal of the gated radiation therapy to determine a current respiratory phase; and controlling the radiation beam on the basis of the correlation relation of the current respiratory phase and a characteristic signal with the external signal. According to one embodiment, setting the gated radiation therapeutic system on the basis of the obtained 4D-CT image comprises setting the gated radiation therapeutic system on the basis of the obtained 4D-CT image and the characteristic signal. The breathing signals for sorting the CT images may either be the characteristic signal, or signals different from the characteristic signal.

It will be understood that controlling the radiation beam on the basis of the correlation relation between the characteristic signal and the external signal may also be realized by using a manner not identical with the above. The contribution of the present invention made over the prior art is reflected in many aspects, and one aspect is to make the gated radiation therapy capable of using the scan result of D4D-CT. This is made possible by using the internal characteristic signal extracted from the CT images to replace the external signal that can only be obtained from the external device. Therefore, though the present application discloses the inventive mode of controlling the radiation beam, other aspects of the gated radiation therapy in the prior art are also suitable for the present invention, including but not limited to, replacing the external signal with the characteristic signal of the present invention merely during the 4D-CT scan, while remaining other aspects of the existing gated radiation therapy unchanged.

According to one embodiment of the present invention, setting the gated radiation therapeutic system on the basis of the 4D-CT image and the characteristic signal comprises projecting radiation merely onto the lesion site within the patient's body when radiation beam is emitted in a particular respiratory phase or its adjacent phase indicated by the characteristic signal because the precise position of the lesion site in the particular phase can be determined through the 4D-CT image. In controlling the radiation beam, it is necessary to determine according to the correlation relation between the characteristic signal and the external signal whether the current respiratory phase is identical with or adjacent to the particular respiratory phase. When the patient is in the particular respiratory phase of the breathing cycle, the lesion site in the body is in a particular position. This particular position is in a therapeutic region (i.e., a region where the radiation reaches). Only in the particular respiratory phase (or adjacent respiratory phase) corresponding to the particular position, can the radiation beam be emitted, and the radiation beam is not emitted in other respiratory phases. Because the region reached by the radiation can be set to be as identical as possible with the size of the lesion site, the area of the normal tissues subjected to the radiation around the lesion site can be minimized. In the gated radiation therapy, it is well known how to set a gated radiation therapeutic system according to the 4D-CT image and the characteristic signal (breathing signals), so the details are not described herein.

It needs to be noted that the current respiratory phase being adjacent to the particular respiratory phase means that the difference between the current respiratory phase and the particular respiratory phase is within N phases, wherein N may be a proper integer taken according to the requirements in a practical application, such as 1, 2, 3, 4, 5, 6, etc. According to one embodiment of the present invention, in the therapeutic stage, after the current external signal has been measured and the current respiratory phase indicated by the external signal has been determined, if, according to the correlation relation between the characteristic signal and the external signal, it is determined that the current respiratory phase is identical with (or adjacent to) the particular respiratory phase when the lesion site indicated by the characteristic signal is in the therapeutic region, then a radiation beam is allowed to be emitted in the current phase. Such a radiation beam can accurately be projected onto the lesion site in the therapeutic region.

In the gated radiation therapeutic stage, it is possible to use external signals provided by different external devices, which include, but are not limited to: real-time position management (RPM) signals provided by a real-time position management (RPM) module, breathing-amount signals provided by a pneusometer, the signals of the height difference of the patient's body surface as the patient breathes provided by an infrared photographic equipment or the signals of the pressure difference caused by the patient's breathing that are provided by a pressure sensor, etc.

According to one embodiment of the present invention, the external signal used in the gated radiation therapeutic stage is a RPM signal provided by a RPM module, wherein the RPM signal is used for controlling the gated phase. Correspondingly, the characteristic signal (or breathing signal) may be based on one of the following parameters extracted from the CT image of a 4D-CT scan, including, but not limited to, air content, lung area, lung density, body area, lung expansion and contraction vectors, the proportion of the lung to the body, etc.

Alternatively, in order to make the extracted characteristic signal to be well related to the RPM signal used in the therapeutic stage, the characteristic signal may be based on two or more parameters mentioned above. Moreover, after the characteristic signal is obtained, the characteristic signal can be used in the gated therapeutic stage. The methods of extracting the breathing signals (or a characteristic signal) based on air content, lung area, lung density, body area, lung expansion and contraction motion vectors, the proportion of the pulmonic cavity to the body, etc. are known in the art, and are not described in detail herein.

It will be understood that the parameters are not limited to the above-mentioned parameters. Actually, other parameters or combinations can also be used in the present invention as long as the characteristic signal is related to a RPM signal.

However, although the characteristic signal obtained through the above parameters can be used for the gated radiation therapy when the RPM signal is used as external signal, because the characteristic signal tends to have phase shift as compared with the RPM signal, it is unable to accurately determine the phase for emitting the radiation beam.

Therefore, according to one embodiment of the present invention, the characteristic signal based on the patient's body surface motion is provided. Particularly, the height of the body surface in a selected region of a particular couch during the 4D-CT scan (e.g., the couch where the lesion site is located) is used. The characteristic signal obtained in this way can well match (or is identical with) the RPM signal. Besides, since the characteristic signal obtained by using the height of the body surface can well match the RPM signals, it can inventively build a bridge between the D4D-CT scan (or A4D-CT scan) and the gated radiation therapy. Even if the breathing signals used in the sorting process after the D4D-CT scan have a relatively large phase shift as compared with the external signal of the gated radiation therapy, it is still possible to accurately determine the phase of emitting the radiation beam.

FIG. 4 shows a method for obtaining a characteristic signal by using a body surface motion according to one embodiment of the present invention. A slice parallel to the X-Z plane of CT system is shown. The boundaries of the light-colored forefront and the dark-colored background in the slice indicate the patient's body surface profile. In the method, at first, a rectangular region (e.g., one that is calculated based on a plurality of CT slices of the particular couch) is determined for the particular couch, then the body surface height in the rectangular region is calculated for each CT slice of the particular couch. Hence the characteristic signal (or breathing signal) can be obtained, which indicates the corresponding relation between the respiratory phase and the body surface height. The rectangular region can ensure that the same region similar to the motion of the RPM module is always used to capture the up-and-down motion of the body surface. It will be understood that the body surface height in the rectangular region includes, but is not limited to a maximum value and an average value of the patient's body surface height in the region.

According to one embodiment of the present invention, a central line of the selected rectangular region along the X axis may coincide with the central line of the patient's body in the X axis, and a central line of the rectangular region along the Y axis passes through the highest position of the body in the Y axis. As for all CT slices, the shape, size and position of the rectangular region may be constant. This can ensure that the same region is always used to capture the up-and-down motion of the body surface so as to extract the characteristic signal identical with the RPM motion rule.

Although FIG. 4 schematically shows a specific rectangular region, the present invention is not limited thereto. Actually, as long as the characteristic signal is related to (e.g., identical to or approximately identical to) the RPM signal, the size, shape and location of the selected rectangular region can be changed. Even a region with other shape (such as rhombus, round, etc.) can also be used.

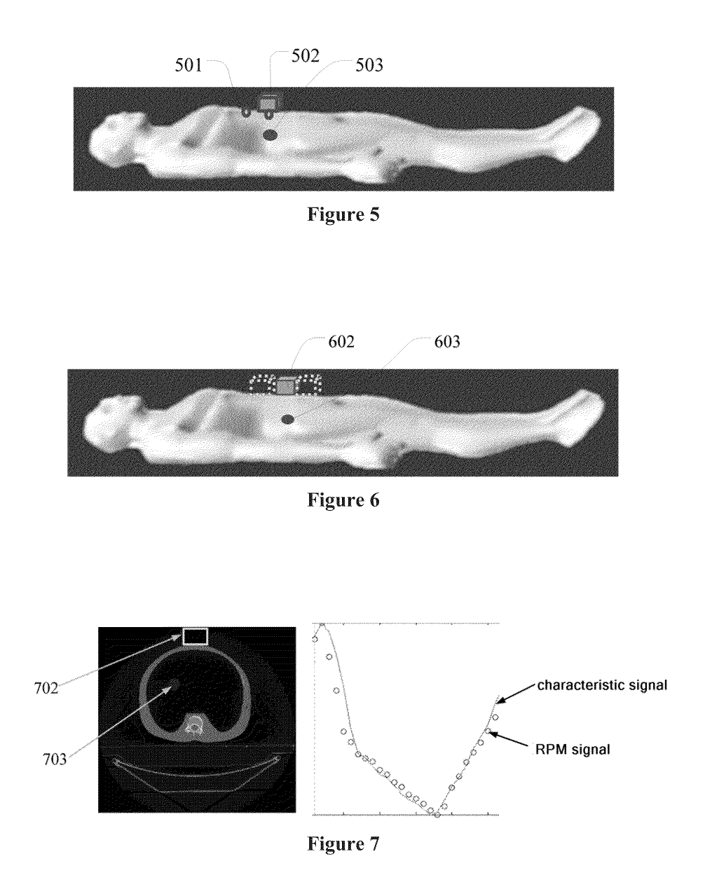

FIG. 5 shows a method for setting a RPM module on the patient's body according to one embodiment of the present invention. The method comprises: fixing a tag 501 to the patient's body when performing a 4D-CT scan in a scan room; determining the position of the tag 501 relative to the lesion site 503 within the body after a 4D-CT image is obtained; fixing a lesion tag on the body surface above the lesion position according to the determined relative position; and setting a RPM module 502 at the lesion site when radiotherapy is performed in a treatment room. In FIG. 5 the small object below the RPM module 502 is the lesion tag. The position of the tag 501 fixed to the patient's body may be the position above the estimated lesion site 503, but the estimated position may be deviated from the actual position. The above method enables the RPM module to be accurately arranged above the body of the lesion site during the treatment.

According to one embodiment of the present invention, when the RPM module is not arranged above the lesion site (or when they are not in the same couch), the characteristic signal still can well match the RPM signal.

FIG. 6 shows the operable region where the RPM module can be set. A RPM module 602 is set right above a lesion site 603. The dotted-line region in FIG. 6 shows the operable region where the RPM module can be set. In one embodiment, the operable region can deviate from the lesion site by at least 9 couches.

FIG. 7 shows the matching condition of the characteristic signal and the RPM signal when a RPM module 702 is positioned right above a lesion site 703. It can be seen that when the RPM module is positioned right above the lesion site, the characteristic signal can well match the RPM signal.

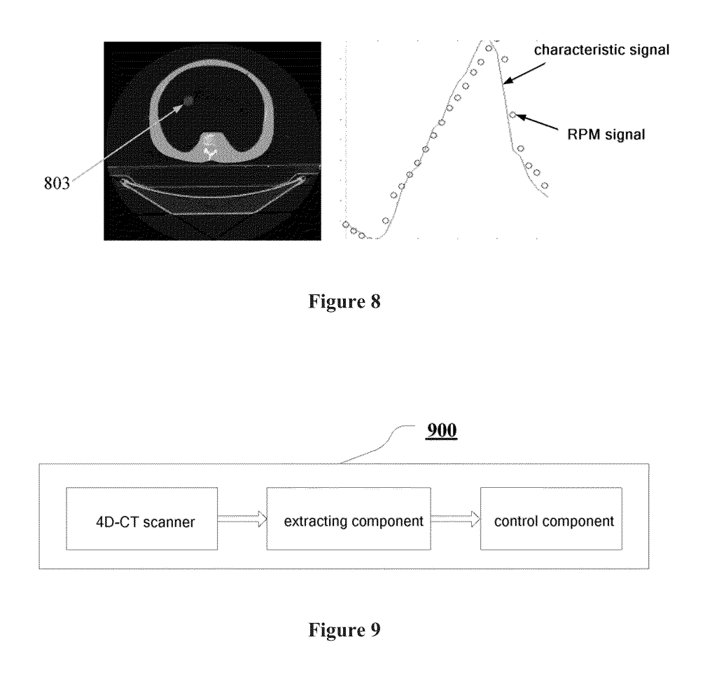

FIG. 8 shows the matching condition of the characteristic signal and RPM signals when the RPM module 802 is positioned in the operation region, but not right above the lesion site. It can be seen that when the RPM module is positioned in the operation region, but not right above the lesion site, the characteristic signal can still well match the RPM signal.

According to one embodiment of the present invention, besides being used as a replacement of an external characteristic signal in the therapeutic stage, the characteristic signal (e.g., breathing signals extracted on the basis of body surface motion, air content, lung area, lung density, body area or their combinations) can also be used in the process of sorting D4D-CT image in the 4D-CT scan stage.

FIG. 9 shows a system for a gated radiation therapy according to one embodiment of the present invention, the system comprising: a 4D-CT scanner for performing a 4D-CT scan to obtain CT images; a characterizing signal extracting component for processing the CT images to extract a characteristic signal, wherein the characteristic signal is associated with respiratory phases of the patient's breathing cycle, and the characteristic signal has a correlation relation with the external signal of the radiation therapy; and a radiation control component for controlling a radiation beam for the radiation therapy on the basis of the correlation relation between the characteristic signal and the external signal.

In the system according to one embodiment, the radiation control component is configured to: sort the CT images to obtain a 4D-CT image; set the gated radiation therapeutic system on the basis of the obtained 4D-CT image and the characteristic signal; measure a current external signal of the gated radiation therapy to determine a current respiratory phase; and control the radiation beam on the basis of the current respiratory phase and the correlation relation of the characteristic signal and the external signal.

In the system according to one embodiment, setting the system for the gated radiation therapy on the basis of the 4D-CT image and the characteristic signal comprises: projecting radiation to only a lesion site of the patient according to the 4D-CT image when the radiation beam is emitted in a particular respiratory phase or its adjacent phase indicated by the characteristic signal.

In the system according to one embodiment, controlling the radiation beam on the basis of the current respiratory phase comprises emitting the radiation beam in the current respiratory phase when the current respiratory phase is identical with or adjacent to the particular respiratory phase. In the system according to one embodiment, the external signal of the gated radiation therapy is a real-time position management (RPM) signal provided by a real-time position management (RPM) module.

In the system according to one embodiment, the characteristic signal indicates patient's body surface motion.

In the system according to one embodiment, the body surface motion comprises: the change of the body surface height in a selected region of a particular couch during the 4D-CT scan.

In the system according to one embodiment, the body surface height is indicated by the maximum height value, average height value in the selected region.

In the system according to one embodiment, the method for a gated radiation therapeutic system comprises: fixing a tag to the patient's body when the 4D-CT scan is obtained, and after the 4D-CT image is obtained, the relative position of the tag and the lesion site is determined; according to the determined relative position, the lesion tag is fixed to the body surface above the lesion site; and when the radiation therapy is carried out, the RPM module is arranged at the lesion tag.

In the system according to one embodiment, the correlation relation between the characteristic signal and the external signal of the gated radiation therapy comprises the characteristic signal matches the external signal.

According to one embodiment, the characteristic signal is used for sorting the CT images to obtain a D4D-CT image.

In the system according to one embodiment, the characteristic signal is obtained from one of the following parameters or their combinations: air content, lung area, lung density, body area, lung expansion and contraction motion vectors, the proportion of the pulmonic cavity to the body, etc.

Those skilled in the art will understand that besides CT, the method for the radiation therapeutic system of the present invention can also be used for other types of radiation therapeutic systems, including, but not limited to: MRI system, linear accelerator (LINAC) system, etc. The present application also discloses a general method for the radiation therapeutic system, comprising: performing a 4D medical scan to obtain images; processing the images to extract a characteristic signal, wherein the characteristic signal is associated with respiratory phases within the patient's breathing cycle, and the characteristic signal has a correlation relation with the external signal of the radiation therapy; and controlling a radiation beam for the radiation therapy on the basis of the correlation relation between the characteristic signal and the external signal.

In the method for the radiation therapeutic system according to one embodiment, controlling the radiation beam on the basis of the correlation relation between the characteristic signal and the external signal comprises: sorting images to obtain a 4D image; setting a radiation therapeutic system on the basis of the obtained 4D image and the characteristic signal; measuring a current external signal of the radiation therapy to determine a current respiratory phase; and controlling the radiation beam on the basis of the current respiratory phase and the correlation relation of the characteristic signal and the external signal.

According to the method for the radiation therapeutic system in one embodiment, setting the radiation therapeutic system comprises: projecting radiation onto only the lesion site of the patient when the radiation beam is emitted in a particular respiratory phase or its adjacent phase indicated by the characteristic signal.

According to the method for the radiation therapeutic system in one embodiment, controlling the radiation beam on the basis of the current respiratory phase comprises: emitting the radiation beam in the current respiratory phase when the current respiratory phase is identical with or adjacent to the particular respiratory phase.

According to the method for the radiation therapeutic system in one embodiment, the external signal of the gated radiation therapy is a real-time position management (RPM) signal provided by a real-time position management (RPM) module.

According to the method for the radiation therapeutic system in one embodiment, the characteristic signal indicates the change of the patient's body surface height in a selected region of a particular couch during the medical scan.

According to the method for the radiation therapeutic system in one embodiment, the method for a gated radiation therapeutic system comprises: fixing a tag to the patient's body when a 4D medical scan is performed; the relative position of the tag and the lesion site is determined after the 4D image is obtained; according to the determined relative position, the lesion tag is fixed to the body surface above the lesion site; and when the radiation therapy is carried out, the RPM module is arranged at the lesion tag.

By using the method for the radiation therapeutic system in the present invention, the cost of the radiation therapy can be reduced and the efficiency of the radiation therapy can be increased.

It will be understood by those skilled in the art that the present invention can be realized in various known ways in this field, including, but not limited to: hardware, firmware, computer program, logical means, etc.

Through the above description and the corresponding drawings, the preferred embodiments of the present invention have been revealed in detail. Besides, though some special terms are used in the text, they are intended to be exemplary only. Those skilled in the art will appreciate that various modifications, equivalent replacements, changes, etc. may be made to the present invention. For example, one step or module in the above embodiments may be divided into two or more steps or modules for implementation, or conversely, two or more steps or functions of modules or means in the above embodiments are put into one step or module for implementation. As long as these changes do not depart from the spirit of the present invention, they should come within the protection scope claimed in the present application. The protection scope of the present invention is defined by the attached claims.

Reference in the specification to “one embodiment” or “an embodiment” of the invention means that a particular feature, structure, or characteristic described in connection with the embodiment is included in at least one embodiment of the invention. Thus, the appearances of the phrase “in one embodiment” in various places throughout the specification are not necessarily all referring to the same embodiment.

This written description uses examples to disclose the invention, including the best mode, and also to enable any person skilled in the art to practice the invention, including making and using any computing system or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A method for a gated radiation therapeutic system, comprising:
performing a 4D-CT scan to obtain CT images;
processing the CT images to extract a characteristic signal, wherein the characteristic signal is associated with respiratory phases in a patient's breathing cycle, and the characteristic signal has a correlation relation with an external signal of a gated radiation therapy; and
controlling a radiation beam for the gated radiation therapy based on the correlation relation between the characteristic signal and the external signal.

2. The method according to claim 1, wherein controlling the radiation beam for the gated therapy based on the correlation relation between the characteristic signal and the external signal comprises:
sorting the CT images to obtain a 4D-CT image;
setting the gated radiation therapeutic system based on the 4D-CT image and the characteristic signal;
measuring a current external signal of the gated radiation therapy to determine a current respiratory phase; and
controlling the radiation beam based on the current respiratory phase and the correlation relation between the characteristic signal and the external signal.

3. The method according to claim 2, wherein setting the gated radiation therapeutic system based on the 4D-CT image and the characteristic signal comprises projecting radiation onto only a lesion site within a patient's body when the radiation beam is emitted in a particular respiratory phase indicated by the characteristic signal.

4. The method according to claim 3, wherein controlling the radiation beam for the gated radiation therapy based on the current respiratory phase and the correlation relation comprises emitting the radiation beam in the current respiratory phase when the current respiratory phase is identical with or adjacent to the particular respiratory phase.

5. The method according to claim 4, wherein the external signal of the gated radiation therapy is a real-time position management (RPM) signal provided by a real-time position management (RPM) module.

6. The method according to claim 5, wherein the characteristic signal indicates a body surface motion of the patient.

7. The method according to claim 5, wherein:
when the 4D-CT scan is performed, a tag is fixed to the patient's body;
after the 4D-CT image is obtained, a relative position of the tag and the lesion site is determined;
according to the determined relative position, a lesion tag is fixed to the patient's body surface above the lesion site; and
when the radiation therapy is carried out, the RPM module is arranged at the lesion tag.

8. The method according to claim 1, wherein the correlation relation between the characteristic signal and the external signal of the gated radiation therapy comprises the characteristic signal matching the external signal.

9. The method according to claim 1, wherein the characteristic signal can be used for sorting the CT images to obtain a D4D-CT image.

10. A system for a gated radiation therapy, the system comprising:
a 4D-CT scanner for performing a 4D-CT scan to obtain CT images;
a characteristic signal extracting component for processing the CT images to extract a characteristic signal, wherein the characteristic signal is associated with respiratory phases in a patient's breathing cycle, and the characteristic signal has a correlation relation with an external signal of a gated radiation therapy; and
a radiation control component for controlling a radiation beam for the gated radiation therapy based on the correlation relation between the characteristic signal and the external signal.

11. The system according to claim 10, wherein the radiation control component is configured to:
sort the CT images to obtain a 4D-CT image;
set the system for the gated radiation therapy based on the obtained 4D-CT image and the characteristic signal;
measure a current external signal of the gated radiation therapy to determine a current respiratory phase; and
control the radiation beam based on the current respiratory phase and the correlation relation between the characteristic signal and the external signal.

12. The system according to claim 11, wherein setting the gated radiation therapeutic system based on the obtained 4D-CT image and the characteristic signal comprises projecting radiation to only a lesion site of the patient when the radiation beam is emitted in a particular respiratory phase indicated by the characteristic signal.

13. The system according to claim 12, wherein controlling the radiation beam based on the current respiratory phase and the correlation relation comprises emitting the radiation beam in the current respiratory phase when the current respiratory phase is identical with or adjacent to the particular respiratory phase.

14. The system according to claim 13, wherein the external signal of the gated radiation therapy is a real-time position management (RPM) signal provided by a real-time position management (RPM) module.

15. The system according to claim 14, wherein the characteristic signal indicates a body surface motion of the patient.

16. The system according to claim 10, wherein the correlation relation between the characteristic signal and the external signal of the gated radiation therapy comprises the characteristic signal matches the external signal.

17. The system according to claim 10, wherein the characteristic signal is used for sorting the CT images to obtain a D4D-CT image.

18. A method for a radiation therapeutic system, comprising:
performing a 4D medical scan to obtain images;
processing the images to extract a characteristic signal, wherein the characteristic signal is associated with respiratory phases in a patient's breathing cycle, and the characteristic signal has a correlation relation with an external signal of a radiation therapy; and
controlling a radiation beam for the radiation therapy on the basis of the correlation relation between the characteristic signal and the external signal.

19. The method according to claim 18, wherein controlling the radiation beam on the basis of the correlation relation between the characteristic signal and the external signal comprises:
sorting the images to obtain a 4D image;
setting the radiation therapeutic system based on the 4D image and the characteristic signal;
measuring a current external signal of the radiation therapy to determine a current respiratory phase; and
controlling the radiation beam on the basis of the current respiratory phase and the correlation relation between the characteristic signal and the external signal.

20. The method according to claim 19, wherein setting the radiation therapeutic system based on the 4D image and the characteristic signal comprises projecting radiation to only a lesion site of the patient when a radiation beam is emitted in a particular respiratory phase indicated by the characteristic signal.
